(19) United States
(12) Patent Application Publication
(10) Pub. No.: US 2006/0141488 A1
(43) Pub. Date: Jun. 29, 2006

(54) Method for stabilizing or preserving nucleic acid by using amino surfactants

(75) Inventors: Tung-Liang Huang, Hsinchu City (TW); Shang-Chi Lin, Rende Township (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsinchu (TW)

(21) Appl. No.: 11/077,015

(22) Filed: Mar. 11, 2005

(30) Foreign Application Priority Data
Dec. 23, 2004 (TW) 093140290

(51) Int. Cl. C12Q 1/68; C07H 21/02; C07H 21/04

(52) U.S. Cl. 435/6; 536/23.1

(57) ABSTRACT

A method for stabilizing or persevering nucleic acids by forming an insoluble ionic complex between nucleic acids and a surfactant in a biological sample, consisting of a step of contacting the biological sample with an isolation reagent comprising amino surfactants of the formula (I): R₁R₂R₃N(O)ₓ, (I), wherein, R₁ and R₂ each independently is H, C1-C6 alkyl group, C6-C12 aryl group, or C6-C12 aralkyl group; R₃ is C1-C20 alkyl group, C6-C26 aryl group or C6-C26 aralkyl group; and x is an integer of 0 or 1. Moreover, the concentration of the amino surfactants in the reagent ranges from 0.001% to 20%. The present invention also relates to a reagent for stabilizing or preserving nucleic acids in a biological sample.

### BACKGROUND OF THE INVENTION

1. Field of the Invention

The present invention relates to a method and a reagent for stabilizing biomaterials in a sample, and more particularly, to a method and a reagent for stabilizing nucleic acid in a biological sample.

2. Description of Related Art

Nucleic acids are known to carry genetic information of an organism. Nowadays, nucleic acids also play important roles in the research fields of molecular biology. According to recent research results, it is known that genetic defects or diseases development of a patient can be deduced from the abnormality or special sequences of nucleic acids of that patient by clinical practice. Thus the goal for preventing disease occurrence can be achieved by detecting the abnormality of nucleic acids and taking necessary remedying steps for treatment before the onset of diseases. To achieve effective detection of abnormality or special sequences of the nucleic acids, the isolation of nucleic acids from an organism, as well as the steps for keeping the genetic information intact are the key subjects for related applications.

Nucleic acids are active molecules, especially RNA. Conventional methods for isolating active molecules of RNA are generally applied with anti-coagulants, for example—EDTA, into the phlebotomized whole blood, and then the sample is kept at 4° C. until the isolation steps can be performed. RNA expression levels would be affected by adding anti-coagulants, changing of temperatures, the periods of storage and the isolating process of leukocytes; these factors increase the difficulties for predicting disease occurrence by RNA expression. To get better results, isolating RNA from whole blood samples must be performed within 24 hours. However, this imperative processing time usually oppresses the medical technicians heavily, especially when large quantities of samples appear to operate.

PAXgene Blood RNA Tube and PAXgene Blood RNA Isolation Kit are developed and commercialized by Qiagen Company, and the two products co-operate for stabilizing and isolating nucleic acids in whole blood. However, the kits are not cost effective, hampering the applications of the kit to routine uses.

In WO2004013155, Goldsborough et al. discloses a method for stabilizing nucleic acids in a biological sample. The main steps for stabilizing nucleic acids in this method are to modify 2′, 3′, and 5′-OH groups of a nucleic acid with a protecting group first, which is to prevent the nucleic acids digestion by nuclease, and then the modified nucleic acids are treated with primary amines to remove the protecting group. The primary amine used here is only for deprotecting the protecting group rather than for forming a complex with nucleic acids.

On the other hand, in US 20040048384 to Augello, Frank A et al. discloses a collection container and method for collecting a predetermined volume of a biological sample, wherein the whole blood sample includes at least one gene induction-blocking agent in an amount effective to stabilize and inhibit gene induction. The stabilizing agent of the gene induction-blocking agent disclosed here is a quaternary amine. More discussion for the related method can be seen in the description of CA 2299119 in which a method for stabilizing and/or isolating nucleic acids is disclosed. The method described here uses at least two quaternary amines or cationic polymers with a phosphor group to precipitate and protect nucleic acids.

Moreover, a novel composition for isolating and/or stabilizing nucleic acids from biological materials is disclosed in US2004014703. The object of the method of US2004014703 is to provide a composition for stabilizing RNA in the presence of tissue, blood, plasma, or serum. The composition comprises a cationic compound like quaternary amine for nucleic acids stabilization.

There is no disclosure about stabilizing nucleic acids with primary amine, secondary amine or tertiary amine. Therefore, it is desirable to provide an improved method to mitigate and/or obviate the aforementioned problems.

### SUMMARY OF THE INVENTION

The present invention provides a method to stabilize nucleic acids in a biological sample with amino surfactants. The mechanisms of nucleic acids stabilization and preservation of the present invention are different from conventional methods with anti-coagulants or a sample refrigerated at 4° C. Surfactants with primary amines, secondary amines and tertiary amines or the mixtures with various ratios of surfactants are used in the present invention, to stabilize nucleic acids by forming an insoluble ionic complex between nucleic acids and a surfactant. The complex protects RNA inside to prevent RNA degradation by RNase, as well as RNA transcription.

The present invention prolongs the periods of stabilization and preservation with simple procedures. The method also can be performed automatically to increase the throughput and expand the application of molecular diagnostic testing with nucleic acid.

To achieve the object, the stabilizing or preserving reagent and the method of the present invention for stabilizing or preserving nucleic acids by forming an insoluble ionic complex between nucleic acids and a surfactant in a biological sample comprises a step of contacting the sample with a stabilizing or preserving reagent consisted of amino surfactants of the formula (I):
R₁R₂R₃N(O)ₓ, (I)

wherein, R₁ and R₂ each independently is H, C1-C6 alkyl group, C6-C12 aryl group, or C6-C12 aralkyl group; R₃ is C1-C20 alkyl group, C6-C26 aryl group or C6-C26 aralkyl group; and x is an integer of 0 or 1.

When the stabilizing or preserving reagent of the present invention contacts with the biological sample, and an insoluble ionic complex is formed between nucleic acids and the surfactant in the stabilizing or preserving reagent. The complex protects RNA inside to prevent RNA degradation by RNase, as well as RNA transcription, hence the nucleic acids in the biological sample are stabilized and preserved.

One of the best embodiments is, R₁ and R₂ each independently is H or C1-C6 alkyl group; and R₃ is C1-C20 alkyl group when x is 0. The amino surfactants of the present invention can be any conventional amino surfactant. Preferably, the amino surfactant of the present invention is selected from the group consisting of dodecylamine, N-methyldodecylamine, N,N-dimethyldodecylamine, N, N-dimethyldodecylamine N oxide and 4-tetradecylaniline. The contact manner of the biological sample and the stabilizing or preserving reagent of the present invention are not limited, and can be a liquid solution or a solid-state composition. To obtain a better mixing result of the sample and the reagent, the preferred embodiment of the present invention reagent is in a manner of liquid solution.

The weight percentage of amino surfactants is not limited. Preferably, the weight percentage of amino surfactants in the solid-state composition is less than 90%, preferably, from 10% to 90%. When in solution form, the concentration of the amino surfactants in the reagent solution preferably ranges from 0.001% to 20%.

The method of the present invention can be performed without any presence of nonionic detergents or acids. However, the use of nonionic detergents, acids or the mixture thereof accompanied with specific amine surfactants might affect the results. Accordingly, the stabilizing or preserving reagent with amino surfactants can selectively further comprise at least one nonionic detergent. The nonionic detergent can be present as a liquid detergent or a solid one in the stabilizing or preserving reagent. The concentration of said nonionic detergent preferably ranges from 0.01% to 20% while the stabilizing or preserving reagent is in a liquid state; the weight percentage of the detergent ranges from 0.01% to 40% while the composition is in a solid state. The nonionic detergent of the present invention can be any conventional one; preferably, the nonionic detergent is polyoxyethylene. More preferably, the nonionic detergent is Tween 20 or Triton X-100, the most preferably, the nonionic detergent is Tween 20.

The stabilizing or preserving reagent with amino surfactants of the present invention can selectively further comprise at least one acid. The acid can be acid buffers or acid agents in a solid-state. The concentration of the acid buffer is less than 1 M. Preferably, the concentration of the acid buffer ranges from 0.01 to 0.5M. The acid can be any conventional one. More preferably, the acid is selected from a group consisting of maleic acid, tartaric acid, citric acid, oxalic acid carboxylic acids and mineral acids. The pH value of the stabilizing or preserving reagent of the present invention can be any value ranging from 1 to 14. Preferably, the pH ranges from 1 to 7, more preferably, the pH ranges from 1 to 5.

The biological sample with nucleic acids used may be cell-free sample material, plasma, body fluids such as blood, serum, cells, leucocyte fractions, sputum, urine, sperm, faeces, smears, aspirates, tissue samples of all kinds, such as biopsies, for example, parts of tissues and organs, food samples which contain free or bound nucleic acids or cells containing nucleic acids as envisaged according to the invention, such as organisms (single- or multi-cell organisms; insects, etc.), plants and parts of plants, bacteria, viruses, yeasts and other fungi, other eukaryotes and prokaryotes, etc.

The term “nucleic acids” for the purposes of the present invention denotes nucleic acids in the wider sense, and thus includes, for example, ribonucleic acids (RNA) and also deoxyribonucleic acids (DNA) in all lengths and configurations, such as double-stranded, single-stranded, circular and linear, branched, etc., and all possible subunits thereof, such as monomeric nucleotides oligomers, plasmids, viral and bacterial DNA and RNA, as well as genomic and non-genomic DNA and RNA from animal and plant cells or other eukaryotes, mRNA in processed and unprocessed form, tRNA, hn-RNA, rRNA, cDNA as well as all other conceivable nucleic acids. Preferably, the nucleic acids of the present invention are DNA or RNA.

Other objects, advantages, and novel features of the invention will become more apparent from the following detailed description when taken in conjunction with the accompanying drawings.

### BRIEF DESCRIPTION OF THE DRAWINGS

FIG. 1 is an electrophoresis result of purified RNA in examples 1-3 of the present invention.

FIG. 2 is an electrophoresis result of purified RNA in examples 4-6 of the present invention.

FIG. 3 is a quantitative RT-PCR result of purified RNA in examples 7-9 of the present invention, which shows the relative gene expression levels in various preservation periods.

### DETAILED DESCRIPTION OF THE PREFERRED EMBODIMENT

The RNA expression level is determined by the quantity and the quality of the extracted RNA after several days by each of the 3 different methods to preserve whole blood samples.

### Example 1

A 10 ml blood collection tube Vacutainer (EDTA K3, Becton Dickinson) is used to collect the whole blood samples. The sample in the Vacutainer is then stored at 4° C. for 0-4 days and the RNA is then isolated after periods of storage.

According to the supplier's handbook, 1 ml of Red Blood Cell Lysis Buffer (Roche Diagnostics GmbH) is added into 500 μl whole blood to purify leukocytes; then 150 μl of buffer RLT (QIAGEN GmbH) is added into leukocytes for cell lysis; 90 μl of ethanol is subsequently added into the sample. The sample is then applied into a centrifuge tube (QIAGEN GmbH) with silica membrane and a centrifugation step is taken.

The silica membrane in the centrifuge tube is washed with 350 μl buffer RW1 (QIAGEN GmbH), and the DNA molecules on the filter are removed with RNase-free DNase Set (QIAGEN GmbH). The silica membrane is then washed again with 350 μl buffer RW1, and twice with 500 μl buffer RPE (QIAGEN GmbH). The RNA molecules on the silica filter are then eluted twice with 40 μl RNase-free water eventually.

### Example 2

In the present example, the collection of whole blood and RNA extraction is performed with PAXgene Blood RNA Validation Kit (QIAGEN GmbH). Blood sample is refrigerated at 4° C. for 0-4 days. After different storage periods, RNA is extracted directly according to the procedures described in the handbook as recommended by the vendor.

### Example 3

In the present example, the whole blood sample for RNA extraction is stored with N-methyldodecylamine to stabilize RNA.

33 μl of fresh whole blood is mixed with 1 ml of a stabilizing solution consisting of 3% (w/v) of N-methyldodecylamine, 5% (v/v) Triton X-100 and 100 mM tartaric acid and refrigerated for 0-4 days at 4° C. To isolate the RNA, the complexes of secondary amine surfactant and RNA are centrifuged at 5000×g for 10 min. The pellet is dissolved in 50 μl distilled water. 100 μl of buffer RLT (QIAGEN GmbH) and 10 μl of Proteinase K (QIAGEN GmbH) are added into the sample and incubated at 55° C. for 10 min. 200 μl of 1-bromo-3-chloropropane is added into the sample and mixed by vortexing. The sample is then centrifuged for 5 min at 10000×g.

The supernatant is then transferred into a new 1.5 ml tube. The sample is mixed with 90 μl of ethanol, and then applied to a spin column containing a silica membrane. The sample mixture is passed through the membrane under centrifugation. The silica membrane is washed with 350 μl of buffer RW1 (QIAGEN GmbH) and DNA molecules are eliminated by using the RNase-free DNase Set (QIAGEN GmbH). The silica membrane is washed with another aliquot of 350 μl of buffer RW1 and twice with 500 μl of buffer RPE (QIAGEN GmbH). Finally, the RNA molecules are eluted twice with the same aliquot of 40 μl of RNase-free water.

### Example 4

In the present example, the whole blood sample for RNA extraction is stored with dodecylamine to stabilize RNA. A stabilizing solution consisting of 0.3% (w/v) of dodecylamine, 1% (v/v) Triton X-100 and 250 mM tartaric acid is prepared, and the pH value is adjusted to pH 3 with NaOH. A 1-ml fresh whole blood is mixed with 3 ml of the stabilizing solution, and then the sample is stored for 0-4 days at 4° C.

To isolate the RNA, the complexes of amine surfactant and RNA are collected by centrifugation. The pellet is dissolved in 150 μl distilled water. 300 μl of buffer RLT (QIAGEN GmbH) and 30 μl of Proteinase K (QIAGEN GmbH) are added into the sample and incubated at 55° C. for 10 min. 200 μl of 1-bromo-3-chloropropane is added into the sample and mixed by vortexing. The sample is then centrifuged for 5 min at 10000×g. The supernatant is then transferred into a new 1.5 ml tube. The sample is mixed with 270 μl of ethanol, and then applied to a spin column containing a silica membrane.

The procedures are subsequently followed in example 3.

### Example 5

In the present example, the whole blood sample for RNA extraction is stored with N,N-dimethyldodecylamine to stabilize RNA. A stabilizing solution consisting of 5% (w/v) of N,N-dimethyldodecylamine, 2% (v/v) Triton X-100 and 140 mM tartaric acid is prepared. A 333-μl fresh whole blood is mixed with 1 ml of the stabilizing solution, and the mixture is frozen for 0-14 days at −20° C. The procedures of RNA isolating are performed following the description in example 3.

### Example 6

In the present example, the whole blood sample for RNA extraction is stored with a stabilizing solution which consisting of 3% (w/v) of N,N-dimethyldodecylamine N oxide, 1% (v/v) Triton X-100 and 125 mM tartaric acid. The sample mixture is stored for 0-14 days at −20° C. RNA in samples of various storage periods is isolated following the procedures described in example 3.

### Example 7

Agilent 2100 Bioanalyzer (Agilent Technologies) is used to analyze 28S/18S rRNA ratios of RNA isolated in examples 1-3. According to the standard ratio approved by those skilled in the art, the 28S/18S rRNA ratio higher than 1.5 means the RNA molecules are intact; on the other hand, the isolated RNA molecules are in good condition when the 28S/18S rRNA ratio is around 2.0. Besides, a good quality of RNA sample shows OD260/280 ratio in the range of 1.9-2.1 that is determined by a spectrophotometer. The methods described above are used to analyze the quality and quantity of RNA samples, and the results are listed in table 1 shown below.

TABLE 1

| | RNA yield | RNA quality | 28S/18S rRNA |
|---|---|---|---|
| Samples | (μg/ml) | (OD 260/280) | ratio |
| Example 1 | 2.39 ± 0.69 | 2.00 ± 0.07 | 0.77 ± 0.08 |
| Example 2 | 4.68 ± 0.68 | 1.94 ± 0.03 | 1.57 ± 0.13 |
| Example 3 | 7.20 ± 0.48 | 1.98 ± 0.14 | 1.83 ± 0.17 |

Apparently, the results of example 3 in table 1 show a higher yield of RNA than example 1 or example 2. RNA yield of 7.20±0.48 μg can be isolated per ml blood with the stabilizing solution of the present invention in example 3. Also, a ratio of OD 260/280 is 1.98±0.14 as well as the 28S/18S rRNA ratio is 1.83±0.17, and both approach the highest quality value of 2.0.

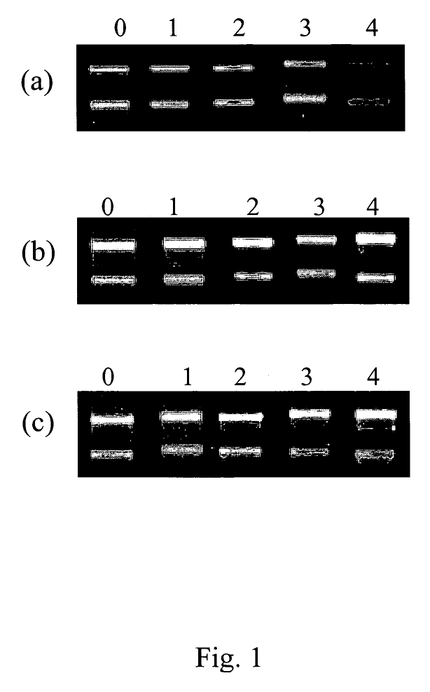

FIG. 1 shows electrophoresis results of purified RNA in examples 1-3, wherein (a) is RNA resulted from example 1; (b) is resulted from example 2 and (c) is from example 3. The numbers 0-4 shown above the figure represent the days of storage. In the results of electrophoresis, the first band of each lane represents 28S rRNA and the second band represents 18S rRNA. Obviously, RNA isolated by the method of the present invention in example 3 has the best quality and quantity than the other two methods in example 1 or 2. The quantity and quality shows no differences between RNA samples from the 4-day storage and from the fresh blood (0-day storage).

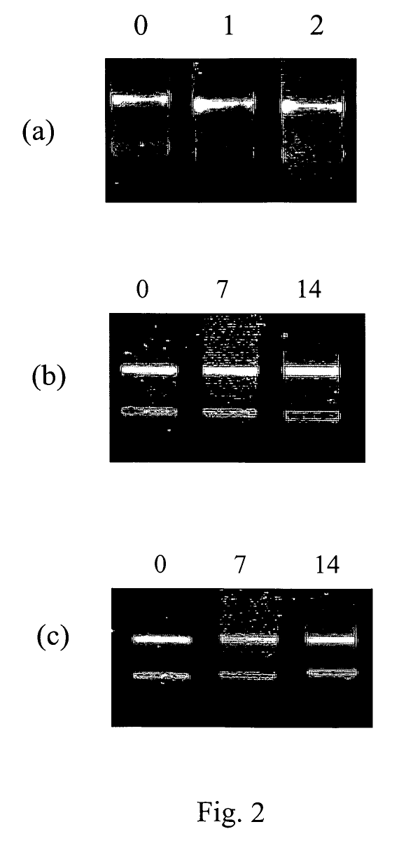

FIG. 2 shows electrophoresis results of purified RNA in examples 4-6, wherein (a) is RNA resulted from example 4 with 0-2 days storage; (b) is resulted from example 5 with 0-14 days storage and (c) is from example 6 with 0-14 days storage. The data from Agilent 2100 Bioanalyzer (Agilent Technologies) are listed in table 2 below. According to the results of table 2 and FIG. 2, RNA isolated from whole blood shows an acceptable condition even after 14 days storage.

TABLE 2

| Samples | Days of storage | 28S/18S rRNA ratio |
|---|---|---|
| Example 4 | 0 days | 1.60 ± 0.14 |
| | 1 day | 1.60 ± 0.00 |
| | 2 days | 1.50 ± 0.00 |
| Example 5 | 0 days | 1.90 ± 0.26 |
| | 7 days | 1.80 ± 0.35 |
| | 14 days | 2.00 ± 0.36 |
| Example 6 | 0 days | 1.70 ± 0.44 |
| | 7 days | 1.53 ± 0.15 |
| | 14 days | 2.10 ± 0.26 |

### Example 8

All procedures in the example are performed as per description in example 1, wherein the whole blood is stored for 0-2 days in 4° C.

### Example 9

All procedures in the example are performed as per description in example 2, wherein the whole blood is stored for 0-2 days in 4° C.

### Example 10

In the present example, 1 ml of whole blood sample for RNA extraction is stored with a 3-ml stabilizing solution consisting of 5% (w/v) of N,N-dimethyldodecylamine and 225 mM tartaric acid (the pH value is adjusted to pH 3.0 with NaOH). The sample mixture is stored for 0-2 days at 4° C. The procedures of RNA isolating are performed after various storage periods following the description in example 4.

### Example 11

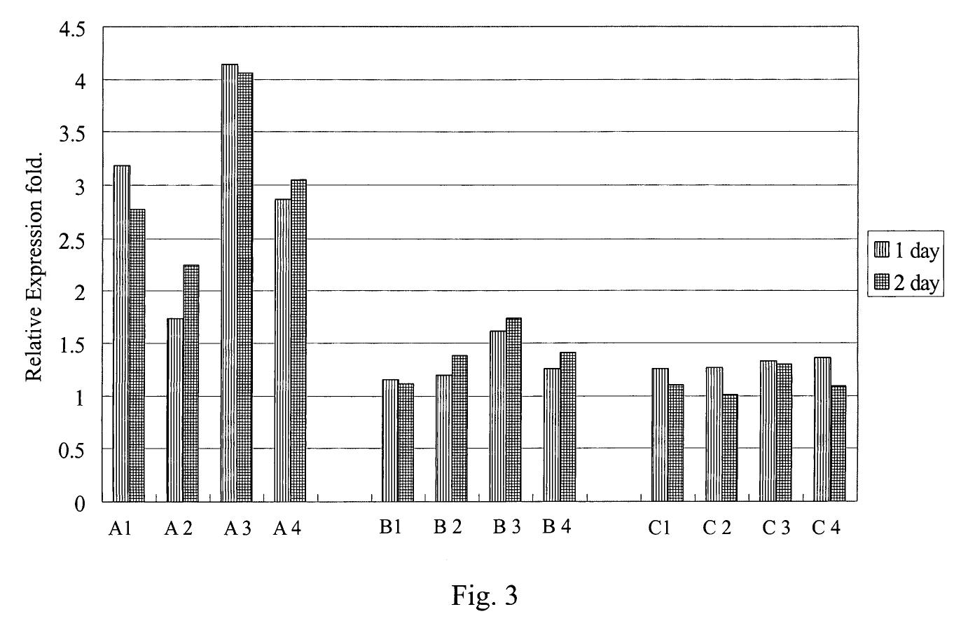

Single strand cDNA molecules are synthesized with the RNA molecules isolated form examples 8-10 by SuperScript II RNase H-Reverse Transcriptase (Invitrogen) according to the procedures described in the handbook as recommended by the vendor. The synthesized single strand cDNA molecules are subsequently performed with TaqMan Universal PCR master mix (Applied Biosystems) and Assays-on-Demand Gene Expression Products (Applied Biosystems), then a real-time PCR process is performed with ABI Prism 7000 Sequence Detection System (Applied Biosystems). The expression levels of 4 genes—ADORA2A, CREB5, NFKB1 and IFNGR1 are determined in different storage periods following the method described above, and the results are shown in FIG. 3.

In FIG. 3, the relative expression fold values represent the ratio of gene expression levels in RNA that are isolated from samples stored at 4° C. over 24-hour or 48-hour compared to no storage. For example, the relative expression fold value of 1 represents that the gene expression level of the isolated RNA molecules after certain period of time of storage is the same as that of 0-hour storage. A1-A4 in FIG. 3 are the results from example 8, wherein A1 is gene ADORA2A, A2 is gene CREB5, A3 is gene IFNGR1 and A4 is gene NFKB1. B1-B4 in FIG. 3 are the results from example 9, wherein B1 is gene ADORA2A, B2 is gene CREB5, B3 is gene IFNGR1 and B4 is gene NFKB1. C1-C4 in FIG. 3 are the results from example 10, wherein C1 is gene ADORA2A, C2 is gene CREB5, C3 is gene IFNGR1 and C4 is gene NFKB1. According to the data in FIG. 3, the relative expression fold value is close to 1 in example 10 and the gene expression levels of the four genes show slight variations. Therefore, example 10 exhibits the best result of preserving nucleic acids in whole blood among these examples.

The embodiments demonstrate that nucleic acids in a biological sample can be stabilized or preserved with several stabilizing solutions containing primary amine, secondary amine or tertiary amine of the present invention. The present invention prolongs the periods of stabilization or preservation with simple procedures according to the data of the examples described above. The method also can be performed automatically to increase throughput and expand the application of molecular diagnostic testing with nucleic acids.

Although the present invention has been explained in relation to its preferred embodiment, it is to be understood that many other possible modifications and variations can be made without departing from the spirit and scope of the invention as hereinafter claimed.

What is claimed is:

1. A method for stabilizing or preserving nucleic acids by forming an insoluble ionic complex between nucleic acids and a surfactant in a biological sample, comprising a step of contacting the biological sample with a stabilizing or preserving composition comprising amino surfactants of the formula (I):
R₁R₂R₃N(O)ₓ, (I),
wherein, R₁ and R₂ each independently is H, C1-C6 alkyl group, C6-C12 aryl group, or C6-C12 aralkyl group; R₃ is C1-C20 alkyl group, C6-C26 aryl group or C6-C26 aralkyl group; and x is an integer of 0 or 1.

2. The method according to claim 1, wherein x is 1, R₁ and R₂ each independently is C1-C6 alkyl, and R₃ is C1-C20 alkyl.

3. The method according to claim 1, wherein x is 0.

4. The method according to claim 1, wherein said amino surfactants are selected from the group consisting of dodecylamine, N-methyldodecylamine, N,N-dimethyldodecylamine, N, N-dimethyldodecylamine N oxide and 4-tetradecylaniline.

5. The method according to claim 1, wherein the concentration of the amino surfactants in the composition ranges from 0.001% to 20%.

6. The method according to claim 1, wherein said composition further comprises at least one nonionic detergent, at least one acid salt, or the mixture thereof.

7. The method according to claim 6, wherein said nonionic detergent is polyoxyethylene.

8. The method according to claim 6, wherein the nonionic detergents in the composition range from 0.01% to 20%.

9. The method according to claim 6, wherein said nonionic detergent is Tween 20 or Triton X-100.

10. The method according to claim 6, wherein said at least one acid salt is selected from a group consisting of maleic acid, tartaric acid, citric acid, oxalic acid, carboxylic acids and mineral acids.

11. The method according to claim 6, wherein the total concentration of said acid salts in the composition ranges from 0.01 M to 1M.

12. The method according to claim 1, wherein the composition is an aqueous medium.

13. The method according to claim 12, wherein the pH of said composition is less than 7.

14. The method according to claim 12, wherein the pH of said composition is less than 5.

15. The method according to claim 1, wherein the composition is in a solid state.

16. The method according to claim 1, wherein said biological sample is selected from the group consisting of whole blood, plasma, serum, urine, tissue and cells.

17. A reagent for stabilizing or preserving nucleic acids in a biological sample comprising amino surfactants of the formula (I):
R₁R₂R₃N(O)ₓ, (I),
wherein, R₁ and R₂ each independently is H, C1-C6 alkyl group, C6-C12 aryl group, or C6-C12 aralkyl group; R₃ is C1-C20 alkyl group, C6-C26 aryl group or C6-C26 aralkyl group; and x is an integer of 0 or 1.

18. The reagent according to claim 17, wherein x is 1, R₁ and R₂ each independently is C1-C6 alkyl, and R₃ is C1-C20 alkyl.

19. The reagent according to claim 17, wherein x is 0.

20. The reagent according to claim 17, wherein said amino surfactants are selected from the group consisting of dodecylamine, N-methyldodecylamine, N,N-dimethyldodecylamine, N,N-dimethyldodecylamine N oxide and 4-tetradecylaniline.

21. The reagent according to claim 17, wherein the concentration of the amino surfactants in the reagent ranges from 0.001% to 20%.

22. The reagent according to claim 17, wherein said reagent further comprises at least one nonionic detergent, at least one acid salt, or the mixture thereof.

23. The reagent according to claim 22, wherein said nonionic detergent is polyoxyethylene.

24. The reagent according to claim 22, wherein the nonionic detergents in the composition range from 0.01% to 20%.

25. The reagent according to claim 22, wherein said nonionic detergent is Tween 20 or Triton X-100.

26. The reagent according to claim 22, wherein the total concentration of said acid salts in the reagent ranges from 0.01 M to 1M.

27. The reagent according to claim 22, wherein said at least one acid salt is selected from a group consisting of maleic acid, tartaric acid, citric acid, oxalic acid, carboxylic acids and mineral acids.

28. The reagent according to claim 17, wherein the reagent is an aqueous medium.

29. The reagent according to claim 27, wherein the pH of said composition is less than 7.

30. The reagent according to claim 27, wherein the pH of said composition is less than 5.
